(19) United States
(12) Patent Application Publication
(10) Pub. No.: US 2011/0257725 A1
(43) Pub. Date: Oct. 20, 2011

(54) Blood Inflating Prosthesis

(75) Inventors: Jeffery Argentine, Petaluma, CA (US); Mark Stiger, Windsor, CA (US); Keith Perkins, Santa Rosa, CA (US)

(73) Assignee: Medtronic Vascular, Inc., Santa Rosa, CA (US)

(21) Appl. No.: 12/763,969

(22) Filed: Apr. 20, 2010

(51) Int. Cl. A61F 2/82

(52) U.S. Cl. 623/1.15; 623/1.24

(57) ABSTRACT

A prosthesis comprises a tubular member that defines an inner lumen and has an inner surface and an outer surface, an outer member secured to the tubular member and covering at least a portion of the tubular member outer surface and forming an outer chamber therewith, and at least one valve in the tubular member to regulate or control fluid flow between the tubular member lumen and the chamber.

### FIELD OF THE INVENTION

The invention relates to grafts suitable for placement in a human body lumen such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., polyester material such as Dacron® fabric, polytetrafluoroethylene PTFE, or expanded polytetrafluoroethylene (ePTFE) or some other polymer) supported by a framework (e.g., one or more stent or stent-like structures) to treat or isolate aneurysms. The framework provides mechanical support and the graft material or liner provides a blood barrier. Approaches for making stent-grafts have included sewing one or more stents or annular metallic spring elements, which may have a sinusoidal configuration, to woven materials, ePTFE, PTFE or Dacron®fabric. Other approaches have included electrospinning the stent structure with a polymer or dip coating. Many stent-grafts have a bare-spring or crown stent attached to one or both of its ends to enhance fixation between the stent-graft and the vessel where it is deployed. The bare-spring or crown stent can be referred to as an anchoring device. In treating an aneurysm, the graft material typically forms a blood impervious lumen to facilitate endovascular exclusion of the aneurysm.

When using a stent-graft, the stent-graft typically is placed so that one end of the stent-graft is situated proximal to or upstream of the diseased portion of the vessel and the other end of the stent-graft is situated distal to or downstream of the diseased portion of the vessel. In this manner, the stent-graft extends through and spans the aneurysmal sac and extends beyond the proximal and distal ends thereof to replace or bypass the dilated vessel wall. The graft material typically forms a blood impervious wall with a lumen therein to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened, and a graft is sutured into position bypassing the aneurysm. The endovascular approach, which has been used to deliver stents, grafts, and stent-grafts, generally involves cutting through the skin to access a lumen of the vasculature. Alternatively, vascular access may be achieved percutaneously via successive dilation at a less traumatic entry point. Once access is achieved, the stent-graft can be routed through the vasculature to the target site where it is deployed.

When using a balloon expandable stent-graft, balloon catheters generally are used to expand the stent-graft after it is positioned at the target site. When, however, a self expanding stent-graft is used, the stent-graft generally is radially compressed or folded and placed at the distal end of a sheath or delivery catheter and self expands upon retraction or removal of the sheath at the target site. More specifically, a delivery catheter having coaxial inner and outer tubes arranged for relative axial movement can be used. The stent-graft is compressed and disposed within the distal end of an outer catheter tube in front of an inner tube. A delivery catheter is typically routed though a vessel, until the end of the catheter (and the stent-graft) is positioned in the vicinity of the intended treatment site. The inner tube is then held stationary while the outer tube of the delivery catheter is withdrawn. The inner tube or stop prevents the stent-graft from moving back as the outer tube is withdrawn. As the outer tube is withdrawn, the stent- graft is gradually exposed from a proximal end to a distal end of the stent-graft, the exposed portion of the stent-graft radially expands so that at least a portion of the expanded portion is in substantially conforming surface contact with a portion of the blood vessel wall. The proximal end of the stent-graft is the end closest to the heart by way of blood flow path whereas the distal end is the end away from the heart during deployment. In contrast and of note, the distal end of the catheter is usually identified to the end that is farthest from the operator while the proximal end of the catheter is the end nearest the operator. Depending on the access location the stent graft and delivery system description may be consistent or opposite. An exemplary stent-graft delivery system is described in U.S. Pat. No. 7,264,632 to Wright et al., the disclosure of which is hereby incorporated herein in its entirety by reference thereto.

When treating aneurysms, stent-grafts typically have stents attached to the graft along the length or a substantial length of the graft to maintain the graft in the desired shape in the aneurismal sac. Grafts also have been provided with epoxy filler to give the graft the desired support and shape. However, there remains a need to improve graft design to provide endolumenal grafts with low delivery profiles and/or provide endolumenal grafts with improved constructions for bridging aneurysms and/or effectively anchoring the grafts in vessels when treating aneurysms.

### SUMMARY OF THE INVENTION

The present invention involves improvements in prostheses.

In one embodiment according to the invention, a prosthesis or implantable endograft device comprises a tubular member that defines an inner lumen, the tubular member having an inner surface and an outer surface; an outer member secured to the tubular member, the outer member covering at least a portion of the tubular member outer surface and forming an outer chamber therewith; and at least one valve in the tubular member to regulate or control fluid flow between the tubular member lumen and the outer chamber.

### BRIEF DESCRIPTION OF THE DRAWINGS

FIG. 1 is a diagrammatic view of a blood inflating prosthesis according to one embodiment of the invention.

FIG. 2 is a diagrammatic view of a blood inflating prosthesis according to another embodiment of the invention.

FIG. 3A is a sectional view of the embodiment of FIG. 1 taken along line 3A-3A before blood inflation.

FIG. 3B is a sectional view of the embodiment of FIG. 1 taken along line 3A-3A in an inflated state.

FIG. 3C is a sectional view showing a variation of the inflatable outer member of FIGS. 3A and 3B.

FIG. 3D is a section view showing another variation of the inflatable outer member of FIGS. 3A and 3B.

FIG. 4A is a side view of filling valve shown in FIG. 2 in a closed position.

FIG. 4B is a further view of the filling valve shown in FIG. 4 in an open position.

FIGS. 5A-C show radial expansion of a blood inflating prosthesis according to one embodiment of the invention where FIG. 5A shows the blood inflating prosthesis deployed in an abdominal aortic aneurysm adjacent to a lower branch vessel prior toinflation; FIG. 5B shows delivery and placement of a balloon catheter into the lower end of the blood inflating prosthesis and inflated so that the balloon blocks blood flow to increase fluid pressure in the blood inflating prosthesis to open the valves and expand the prosthesis; and FIG. 5C shows the prosthesis in an inflated state where it fills the aneurismal sac and conforms with the inner wall of the aneurysm and the balloon catheter removed.

FIG. 5D shows another blood inflating prosthesis embodiment according to the invention where the inflated graft does not completely fill the aneurismal sac.

FIG. 5E1 shows another blood inflating prosthesis embodiment according to the invention, which is a variation of the embodiment of FIG. 1.

FIG. 5E2 shows another blood inflating prosthesis embodiment according to the invention, which is a variation of the embodiment of FIG. 2.

FIG. 5E3 shows the embodiment of FIG. 5E1 deployed and in an inflated state.

FIG. 6 illustrates another blood inflating prosthesis embodiment according to the invention, which is another variation of the embodiment of FIG. 1 with an alternative valve configuration.

FIG. 6A is a sectional view taken along line 6A-6A in FIG. 1.

FIG. 6B illustrates the sectional view of FIG. 6A during inflation with the depicted valve open.

FIG. 6C illustrates the sectional view of FIG. 6A inflated with the depicted valve closed.

FIGS. 7A-C show another valve configuration embodiment according to the invention, where FIG. 7A diagrammatically shows the valve attached to a portion of a tubular member, FIG. 7B shows the valve open during inflation, and FIG. 7C shows the valve closed after inflation.

FIG. 8 illustrates another blood inflating prosthesis embodiment according to the invention.

FIGS. 9-11 diagrammatically show a method of assembling an expandable prosthesis according another embodiment of the invention where FIG. 9 shows an outer member made from a sheet of material wrapped around and secured to the leg portions of a bifurcated prosthesis, which can be a stent-graft, FIG. 10 is a sectional view taken along line 10-10 in FIG. 9, and FIG. 11 shows the outer member everted with its other end secured to the main body portion of the tubular member of the prosthesis to form an expandable chamber.

FIG. 12 is a perspective view of another expandable outer member embodiment according to the invention.

### DETAILED DESCRIPTION

The following description will be made with reference to the drawings where when referring to the various figures, it should be understood that like numerals or characters indicate like elements.

In one embodiment according to the invention, a blood inflating prosthesis or implantable endograft device is constructed for radial inflation with the patient's own blood. The prosthesis has one or more valves (e.g., one-way valves) that allow one or a plurality of chambers or fillable spaces of the prosthesis to be filled with the blood when the prosthesis is pressurized. Pressurization can be achieved by inflating a balloon in a distal part of the prosthesis and using the patient's own blood pressure to open the valve(s) to fill the one or plurality prosthesis chambers or fillable spaces. When the one or a plurality of chambers have been filled, the balloon is deflated and removed. Since the blood pressure and pressure inside the chamber or chambers is about the same after inflation, the blood typically does not transmigrate back into the blood flow. Using the patient's blood to fill the prosthesis chamber(s) can be safer than using other fill materials because the patient's own blood typically would not be rejected. The prosthesis can have only a single sac shaped chamber. Alternatively, the prosthesis can have a plurality of separate expandable or inflatable chambers that form a plurality of expandable or inflatable ribs.

In some embodiments, the filled chamber or chambers provide sufficient structural integrity to eliminate the need for stents in the region of the chambers. In one variation, one or more coagulating agents is provided in the chamber to assist the blood in clotting and/or hardening to maintain the shape of the prosthesis and provide structural support. With this construction, the region of the prosthesis that the fillable chamber surrounds can be stentless, which can reduce cost and/or reduce the delivery profile or diameter of the prosthesis.

In some embodiments, the prosthesis chamber fills in such a manner that the prosthesis expands or enlarges to fill the aneurismal sac and/or conform to the inner wall of the aneurysm. In this manner, the prosthesis or endolumenal graft device can be effectively anchored in the vessel where the aneurysm is being treated to minimize or eliminate prosthesis migration. Accordingly, this construction can minimize or eliminate the need for anchoring with mechanisms such as barbs or other non-stent-like anchoring mechanisms. It also can minimize or prevent lateral movement of the prosthesis within a large aneurysmal sac. Lateral movement can destabilize the fixation and/or seal between the proximal or distal end of the prosthesis and the vessel wall. The blood inflating prosthesis also may allow one to provide sufficient prosthesis fixation where the landing zone adjacent to the aneurysm is small (e.g., less than about 10 mm). The filled aneurysm and/or conformance aspect also can limit exposure of the aneurysmal sac to circulatory pressures that can result from endoleaks at the proximal or distal end of the prosthesis/or minimize or eliminate blood flow from collateral vessels into the aneurysmal sac. Small voids between the prosthesis and aneurysmal sac can fill with blood that coagulates to further assist in minimizing or eliminating blood flow from collateral vessels into the aneurysm and/or prosthesis fixation.

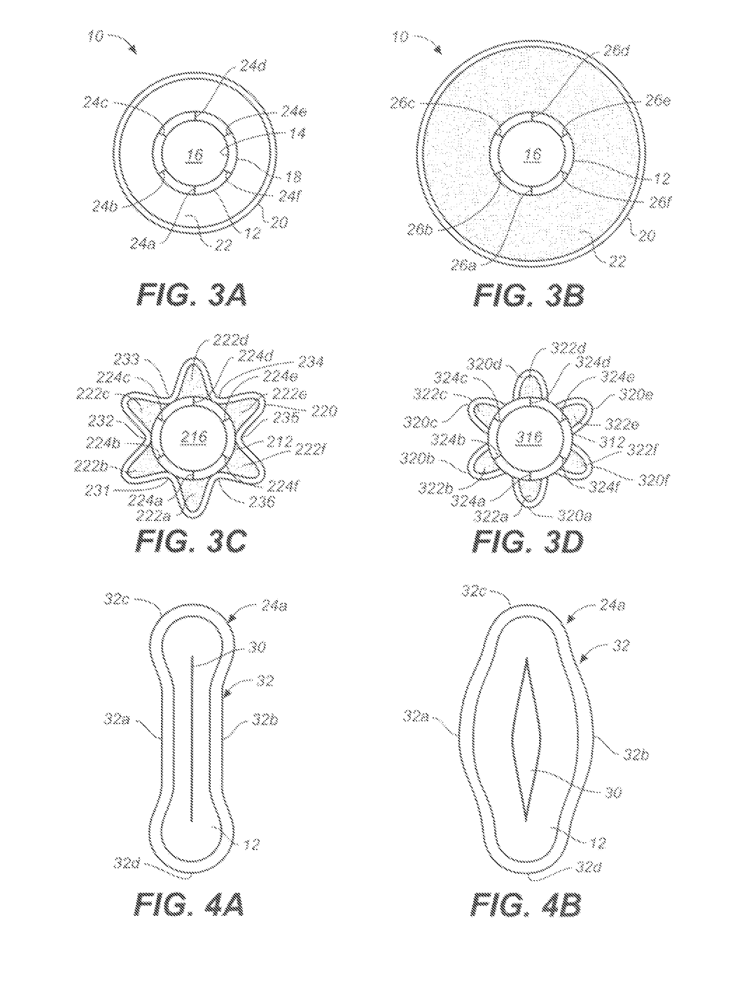

Referring to FIG. 1, one embodiment of a blood inflating tubular prosthesis or implantable endolumenal graft is shown and generally designated with reference numeral 10. Tubular prosthesis 10 generally comprises a tubular member 12, which can be a bifurcated graft as shown or a non-bifurcated single lumen graft depending on the application. A bifurcated prosthesis in the form of a stent-graft is suitable for abdominal aortic aneurysms that are below the renal arteries. Tubular member 12, which typically has a single wall construction, has an inner surface 14 that defines an inner lumen 16 and an outer surface 18 (see e.g., FIG. 3A). Tubular member or graft 12 defines a blood flow passage and can comprise any suitable graft material (e.g., polyester material such as Dacron® polyester (polyethylene terephthalate) fabric, polytetrafluoroethylene PTFE, or expanded polytetrafluoroethylene (ePTFE) or the like used by those skilled in the art of stent graft engineering and manufacturing). An outer member or sac 20 is secured to tubular member 12 such that it covers at least a portion of outer surface 18 of tubular member 12 to form outer radially expandable or inflatable chamber 22 therewith (see e.g., FIG. 3A). In this manner, tubular member 12 and outer member or sac 20 form an radially expandable bladder. In the illustrative embodiment, stents are not required in the region of tubular member 12 that is covered by outer member or sac 20 since the sac when filled will provide support for the tubular member 12 (i.e., tubular member 12 can be stentless or without stents or stent-like graft support members, which do not include the sac filling fluid/blood described herein or treated fluid/blood described below) in the region or portion of tubular member 12 located radially inward of outer member or sac 20 or covered by outer member or sac 20 or the region where outer member 20 covers tubular member 12 and is inflated). In one variation, one or more coagulating agents such as an autologous platelet gel (autologous platelet rich plasma combined with a thombogenic agent) and/or a bioactive biopolymer (e.g. polyglycol lactic acid (PGLA)), which bioactive polymer increases the rate of the conversion of thrombus into fibrocelluar tissue, which is more robust than thrombus and has a greater structural integrity than thrombus, is placed in the outer chamber 22 prior to use so that the blood coagulates and/or hardens in outer chamber 22 to provide additional structural support for tubular member 12 and outer member 20. The bioactive polymer also may enhance biological fixation to promote desirable tissue ingrowth in outer member 20. Although the addition of one or more coagulating agents and/or a bioactive biopolymer has been described regarding blood inflating prosthesis 10 and outer chamber 20, it should that such agent(s) and/or biopolymer can be added to any of the outer chambers described herein.

The absence of stents in the region of tubular member 12 that outer member 20 covers or the region of tubular member 12 that the inflated or inflated portion of outer member 20 covers reduces the delivery profile or diameter of the prosthesis. It also can reduce production costs. For example, stents often are sewn to the graft material and a reduction or elimination of this step can significantly reduce manufacturing time. Further, reducing components such as stents reduces material costs.

Outer member 20 is adapted to at least substantially fill or to at least substantially conform to the enlarged volume of the aneurysm. Outer member 20 can be shaped for the aneurysm of a particular patient using imaging and computer aided design and fabrication techniques. Alternatively, a collection of different outer member geometries and sizes can be provided for the physician to select one based on the size and configuration of a patient's aneurysm. Outer member 20 typically will be formed from a non-compliant material such as parylene, Dacron, PET, PTFE, a compliant material such as silicone, polyurethane, latex, or a combination of these materials. Outer member 20 can be formed partially or entirely from non-compliant material to enhance conformance of outer member 20 to the inner surface of the aneurysm. This may be especially desirable when outer member 20 has been individually designed and/or sized for the patient being treated. Outer member 20 also can be a generally compliant and expandable blood impermeable material such as Pebax® (PEBA), C-Flex® (TPE), Tecothane® (TPU), or Carbothane® polyurethane carbonate, and any materials of the like, that which when unstressed collapse so as to be disposed in close proximity to tubular member 12.

Outer member 20 also may consist of a single layer or may comprise multiple layers, which are laminated. Different layers may comprise different materials including both compliant and/or non-compliant materials. Outer member 20 also may be reinforced with braid reinforcement layers, filament reinforcement layers, or other reinforcement materials.

In the illustrative embodiment, the ends 20a and 20b of outer member 20 are secured to tubular member 12 to form a fluid tight seal such that chamber 22 can be filled or inflated. In this embodiment, outer member 20 is a pre-molded thin walled cylindrical component having an open end and a closed end, which is closed except for two openings formed therethrough. At the open end, end 20a is annular and forms an opening for receiving the large or single lumen portion of tubular member 12. The closed end comprises disk shaped end 20b having two openings formed therethrough for receiving leg 10a and truncated leg 10b of tubular member 12. The annular portions of end 20b that define the openings are secured to legs 10a and 10b to form a fluid tight seal therebetween using any suitable sealing means such as thermal/heat sealing, adhesive bonding or gluing, or any combination thereof. A fluid tight seal between end 20a and tubular member 12 also is provided using any of the sealing means described above.

Valves 24a-f and 26a-f are provided in the wall of tubular member 12 to allow one-way fluid flow between inner lumen 16 and outer chamber 22 or from inner lumen 16 to chamber 22. Accordingly, these valves provide fluid communication between lumen 16 (as well as blood in lumen 16 during use) and outer chamber 22 when the valves are open. When the pressure is equalized between inner lumen 16 and outer chamber 22, flow will also subside and these valves will begin to close. When the pressure in outer chamber 22 begins to increase above the pressure of inner lumen 16 (for any number of reasons), the valve will shut automatically to prevent fluid from escaping from outer chamber 22 to inner lumen 16. The action of valves closing thus maintains the volume of the outer chamber 22 and therefore maintains the structural shape and integrity of the inflated prosthesis.

Regarding the valve arrangement, a plurality of valve sets can be used where the valves in each valve set are circumferentially spaced around tubular member 12 and each valve set is longitudinally spaced from one another. One example of such a configuration is shown in FIGS. 1 and 3A and 3B where six valve set 24a-f(FIGS. 1 and 3A) and six valve set 26a-f (FIGS. 1 and 3B) are longitudinally spaced from one another with each valve in valve set 24a-f longitudinally aligned with a valve in valve set 26a-f. In one alternative, the valves in the two valve sets can be arranged with the valves in one valve set circumferentially staggered from the valves in the other valve set (i.e., the valves of the two valve sets are not aligned). In a further alternative, the valves can be arranged in one or more spirals. Other valve configurations and numbers also can be used based on the desired filling rates and expandable sac configuration. For example, a single valve can be used. In another example, six valves can be circumferentially spaced around tubular member 12 in the central region of tubular member 12.

Tubular member 12 of prosthesis 10 has a main body portion above the bifurcation and a first leg 10a and a second truncated leg 10b for receiving leg 50. Prosthesis can be provided with undulating annular bare spring or crown stent 51, which also can be a suprarenal stent, and undulating annular stents S2a-c. Such bare springs or crown stents can serve as anchoring devices. Leg 50, which is to be secured to prosthesis 10 as is known in the art, is a tubular graft, which can include any suitable number of undulating annular stents such as indicated with reference characters S3a-c. The stents can be attached to tubular member (or tubular graft) 12 and tubular graft leg 50 with stitching or any other conventional means. The stents generally provide a mechanical support or framework for the tubular members or grafts, which provide a pathway for blood circulation. As shown in FIG. 1, the main body portion above the bifurcation unsupported by stents except for the crown stent. The embodiment of FIG. 1 also shows the portion of the prosthesis aligned or covered by outer member 20 to be unsupported by stents.

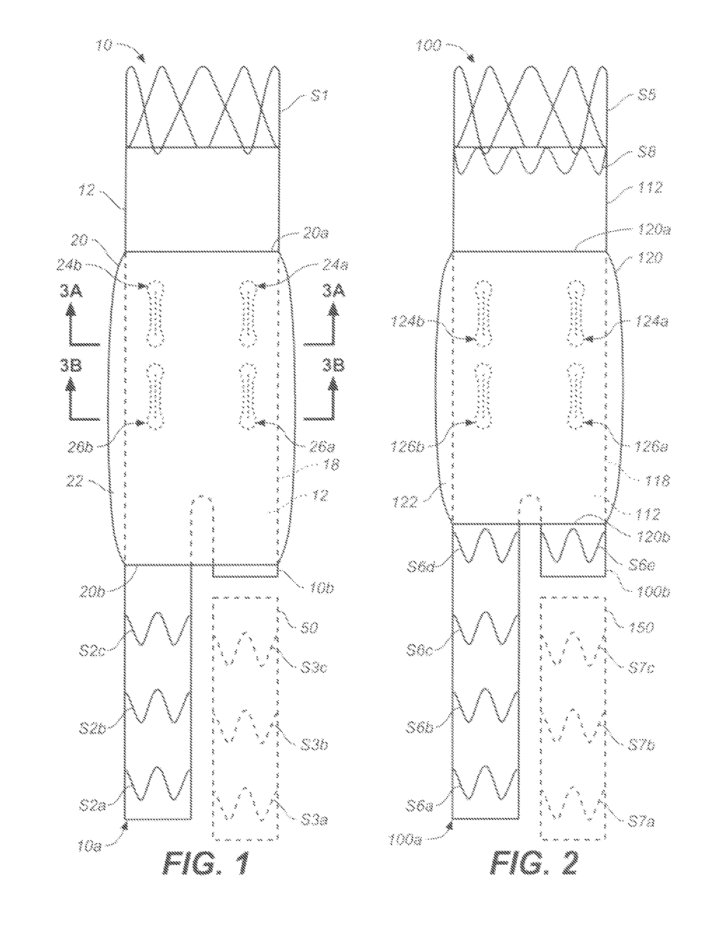

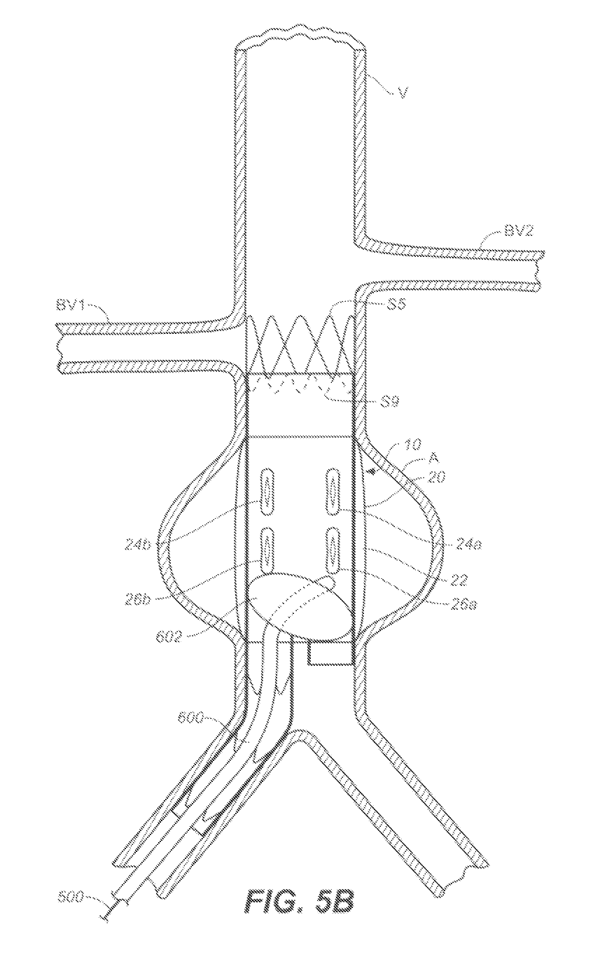

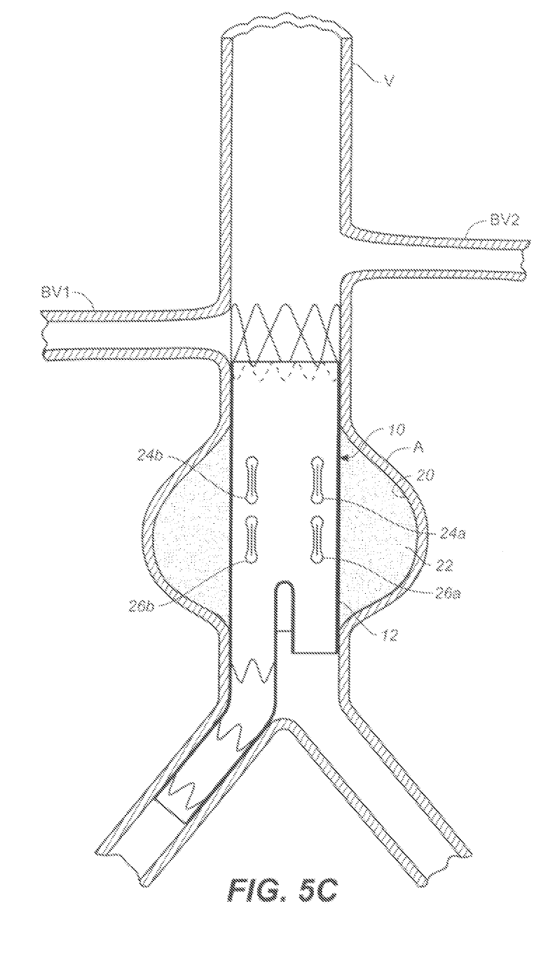

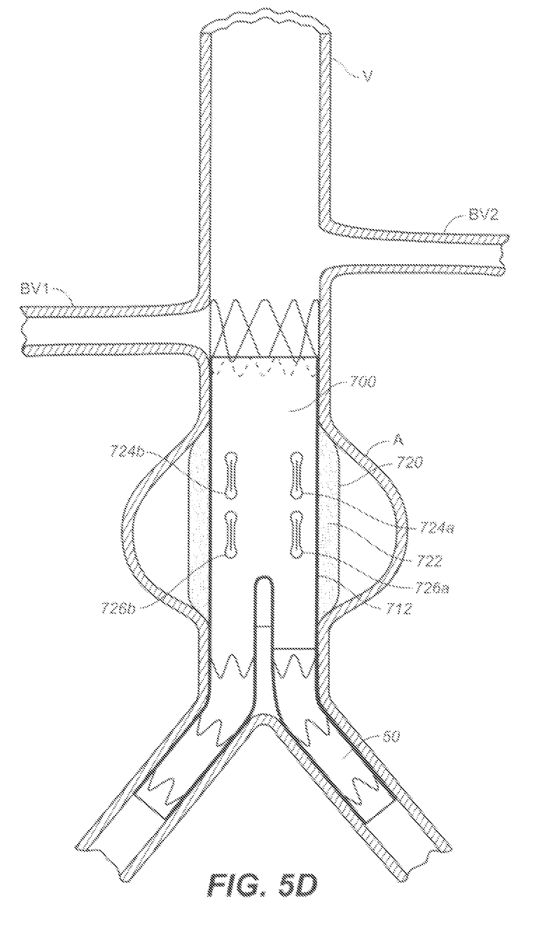

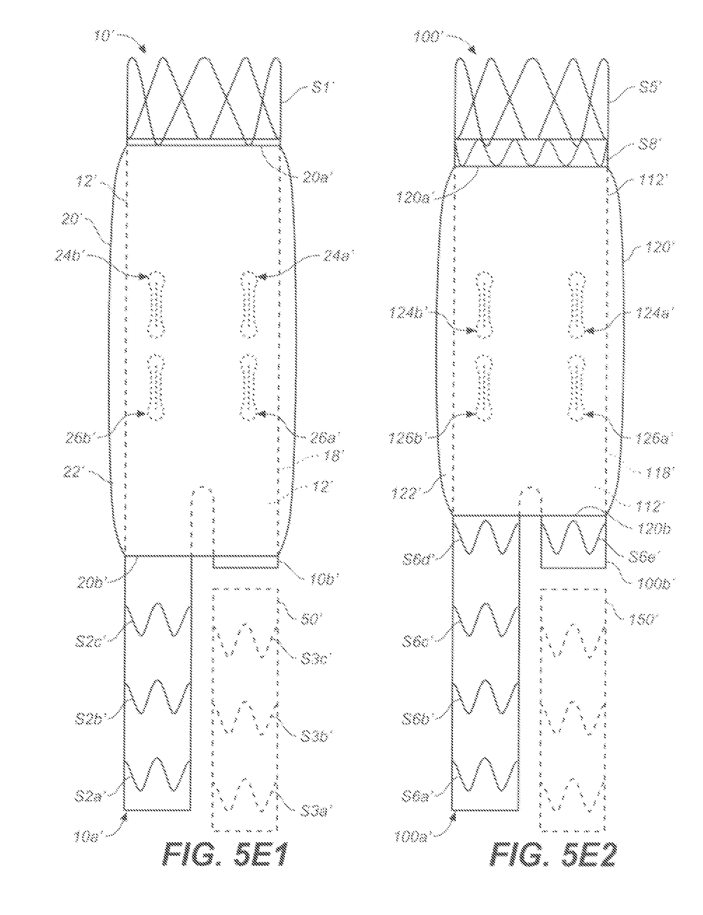

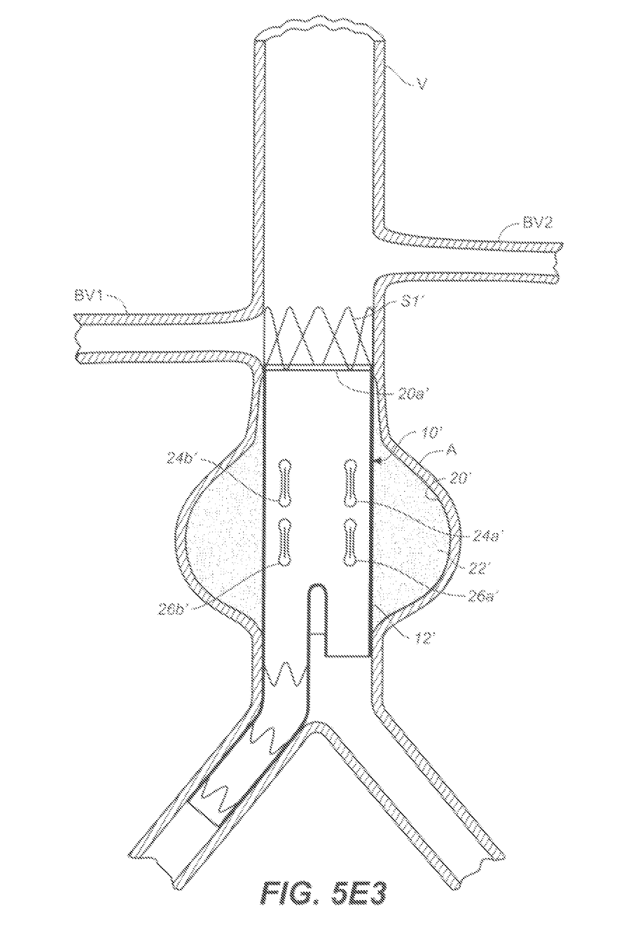

FIG. 2 illustrates another blood inflating prosthesis 100 which is the same as prosthesis 10 with the exception that outer member 120 does not extend as far below the bifurcation and two additional stents S6d and S6e are used in the uncovered region. The shorter outer member shown in FIG. 2 terminates closer to the bifurcation as compared to that in FIG. 1, could allow better flexibility and/or articulation of the truncated leg (contralateral leg) 100b for either cannulation and/or axial alignment with the limb segment of the implant 150. Otherwise elements 100a (leg), 100b (leg), 112, 114, 116, 118, 120, 120a, 120b, 122, 124a,b and 126a, b are the same as elements 10a, 10b, 12, 14, 16, 18, 20, 20a, 20b, 22, 24a,b and 26a,b and although not shown in FIG. 2, prosthesis 100 includes two six valve sets as shown in FIGS. 1, 3A, and 3B in connection with prosthesis 10. Prosthesis 100 includes undulating annular bare spring or crown stent S5, which can be a suprarenal stent, undulating annular stents S6a-e, and optional undulating annular spring element or stent S8, which can be optionally incorporated into prosthesis 10 as well as shown in FIG. 5A-C where it is designated with reference numeral S9, which has the same construction as stent S8. Leg 150, which is to be secured to prosthesis 100 as is known in the art, is a tubular graft, which can include any suitable number of undulating annular stents such as indicated with reference characters S7a-c. The stents can be attached to tubular member or graft 112 and tubular graft leg 150 with stitching or any other conventional means. The stents generally provide a mechanical support or framework for the tubular members or grafts, which provide a pathway for blood circulation.

Referring to FIGS. 3A and 3B, prosthesis 10 is shown before radial inflation and after radial inflation. More specifically, FIG. 3A shows outer member 20 in an uninflated configuration where valves 24a-f and 26a-f are closed state. When pressure in tubular member 12 sufficiently exceeds that in outer chamber 22, the valves open and blood flows into chamber 22 and expands outer member or sac 20. That is, the pressure differential opens the valves so that the blood flows from an area of higher pressure to an area of lower pressure. When the pressure in inner lumen 16 of tubular member 12 and the pressure in outer chamber 22 are about equal, spring elements 32 close the valves as will be described in further detail below. FIG. 3B shows outer chamber 22 filled with blood, outer member 20 inflated, and the valves closed.

FIG. 3C is a sectional view showing a variation of the expandable outer member of FIGS. 3A and 3B illustrating a ribbed configuration in an inflated state. In this variation, outer member 220 corresponds to outer member 20 with its ends sealingly secured to tubular member 212 in the same as outer member ends 20a and 20b are sealing secured to tubular member 12. However, outer member 220 also is sealingly secured to tubular member 212 along longitudinal seams 231-236, which extend the entire length of outer member 220 to form a plurality of fillable spaces or chambers 222a-f, which are shown in a filled state. Longitudinal seams or attachments 231-236 can be formed using any suitable means such as heat fusion, stitching, adhesive bonding, or any combination thereof. Valves 224a-f, which can have the same construction as valve 24a, which will be described in more detail below, facilitate regulation of fluid flow from lumen 216 of tubular member 212 to fillable spaces or chambers 222a-f.

FIG. 3D is a sectional view showing another variation of the expandable outer member of FIGS. 3A and 3B illustrating another ribbed configuration in an inflated state. In this variation, a plurality of discrete or separate outer members 320a-f are circumferentially spaced around and sealingly secured to tubular member 312 to form therewith a plurality of longitudinally extending fillable spaces or chambers 322a-f. Valves 324a-f, which can have the same construction as valve 24a, which will be described in more detail below, facilitate regulation of fluid flow between lumen 316 and tubular member 312 or from lumen 316 of tubular member 312 to fillable spaces or chambers 322a-f.

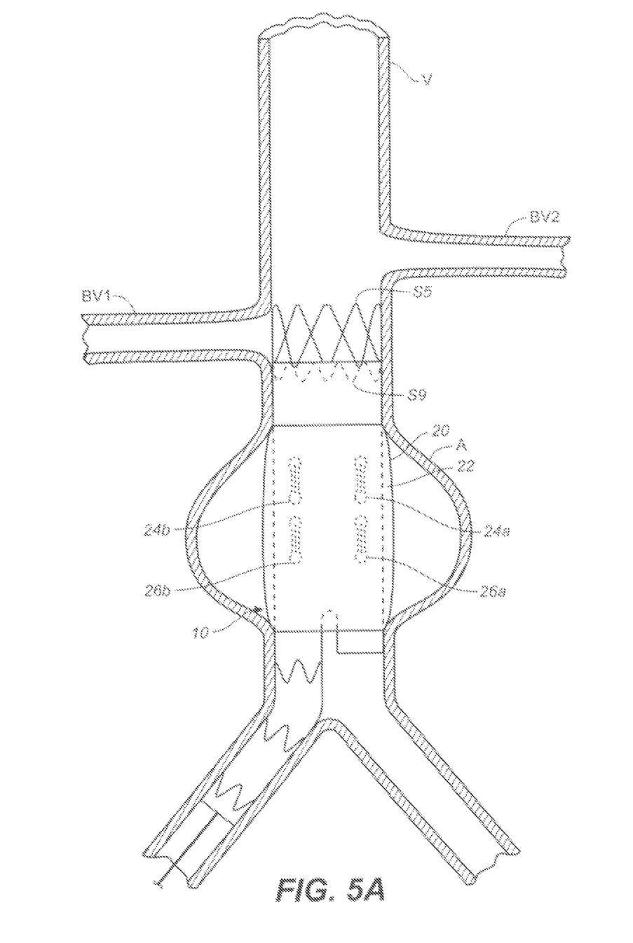

Referring to FIGS. 4A and 4B, one valve embodiment will be described in more detail. Since all of the valves can have the same construction, one valve (valve 24a) is shown in FIGS. 4A and 4B for simplification. Valve 24a is a one-way valve and is formed by a portion of inner tubular member 12, an openable portion of tubular member 12 (which corresponds to slit 30, which is formed through the wall of tubular member 12), and a spring element 32 or valve member configured and arranged to urge slit 30 closed as shown in FIG. 4A. In the illustrative embodiment spring element 32 comprises a nitinol wire that is preshaped in the closed loop configuration shown in FIG. 4A, where it has a central section with parallel portions 32a and 32b that extend into rounded enlarged end portions 32c and 32d at opposite ends thereof. Further, spring element 32 can be made from shape memory material and provided with a preshaped “memory set” configuration as shown if FIG. 4A and as is known in the art. For example, spring element 32 can be placed in the desired shape (e.g., that shown in FIG. 4A) and heated for about 5-15 minutes in a hot salt bath or sand having a temperature of about 480-515° C. It can then be air cooled or placed in an oil bath or water quenched depending on the desired properties. Spring element 32 can be made from other materials as well such as medical grade stainless steel.

Spring element 32 is configured and arranged to expand and allow slit 30 to open (FIG. 4B) when the pressure inside tubular member 12 is sufficiently greater than the pressure in outer chamber 22 and to hold slit 30 closed or close slit 30 (FIG. 4A) when such a pressure differential is not present. In use, a balloon can be used to block blood flow through the prosthesis to create a positive pressure of typically about 150mm Hg in the inner lumen 16 and since the pressure in outer chamber 22 initially is about zero, the pressure differential opens the valves. This will be described in more detail below with reference to FIG. 5B. After outer chamber 22 is filled, tubular member 12 can be held open by blood flow therethrough. However, the structural characteristics of outer member 20 and semi-rigid outer chamber 22 after filling can be more rigid if one or more anticoagulant agents and/or a bioactive biopolymer were present in outer chamber 22 before filling as described above.

Referring to FIGS. 5A-C, one method of inflating a blood inflating prosthesis will be described with reference to blood inflating prosthesis 10 for purposes of example. It should be understood, however, that any of the blood inflating prostheses described herein can be used in a similar manner. FIG. 5A depicts the blood inflating prosthesis 10 deployed and positioned in an aneurysm “A” below branch vessels “BV1” and “BV2,” which branch from vessel “V.” At this stage, the prosthesis is held in place with suprarenal stent S5 and leg stents S2a,b (FIG. 1). Optional annular spring element or biasing stent S9 also can assist in prosthesis fixation if used. In this example, vessel “V” corresponds to the aorta, the branch vessels correspond to the renal arteries, and the aneurysm is an abdominal aortic aneurysm. Blood inflating prosthesis 10 can be delivered and deployed at the target site with any suitable endolumenal graft delivery catheter as would be apparent to one of ordinary skill in the art. Examples of delivery apparatus that can be used are described in U.S. Pat. No. 7,264,632 to Wright et al., which issued Sep. 4, 2007 and is entitled Controlled Deployment Delivery System, the disclosure of which is hereby incorporated herein in its entirety by reference thereto, or co-owned U.S. patent application Ser. No. 12/426,011 to Stiger et al., which was filed Apr. 17, 2009 and is entitled Stent Graft Restraining Mechanism for a Delivery System (Catheter), the disclosure of which is hereby incorporated herein in its entirety by reference thereto. The delivery catheter is tracked over guidewire 500 using conventional techniques.

Referring to FIG. 5B, any suitable balloon catheter, which is generally indicated with reference numeral 600, is delivered through the femoral artery and tracked over guidewire 500 to the prosthesis and positioned with balloon 602 adjacent the bifurcation or below the valves as shown. Balloon 602 is then inflated so that the balloon blocks blood flow through the prosthesis to increase fluid pressure in inner lumen 16 and open the valves to radially expand outer member 20 and expand chamber 20 (FIG. 5B). Typically the balloon will create a positive pressure of about 150 mm Hg in the lumen 16 of tubular member 12 and since the pressure in outer member 20 initially is about zero, the pressure differential opens the valves. In this regard, the patient's own blood expands outer member 20 to expand the prosthesis. Referring to FIG. 5C, the prosthesis is shown in an inflated state where it at least substantially fills the aneurysmal sac and at least substantially conforms with the inner wall of the aneurysm. When the aneurismal sac is filled and the pressure differential between lumen 16 of tubular member 12 and outer chamber 122 is insufficient to maintain the valves open, the spring elements close the valves (see FIGS. 3B and 5C). After the valves are closed, the balloon catheter is removed. In the illustrative embodiment, the inflated prosthesis is barbless. Contralateral leg 50 is then inserted in and secured to truncated leg 10b (FIG. 1).

Referring to FIG. 5D, another blood inflating prosthesis embodiment is shown in an inflated state where the inflated prosthesis 700 does not completely fill the aneurismal sac. Blood inflating prosthesis 700 is the same as blood inflating prosthesis 10 except that in contrast to inflatable outer member 20, blood inflating prosthesis 700 has an annular expandable outer member 720 that does not expand or expand to substantially fill the aneurismal sac. Otherwise elements 712, 714, 716, 718, 720, 722, 724a,b and 726a,b are the same as elements 12, 14, 16, 18, 20, 22, 24a,b and 26a,b and although not shown in FIG. 5E, prosthesis 700 includes two six valve sets as shown in FIGS. 1, 3A, and 3B in connection with prosthesis 10. Although similar anchoring and/or leak resistance is not provided as compared to inflated prosthesis 10 as shown in FIG. 5D, the blood filled outer member provides support structure to tubular member 712. To this end, one or more blood coagulating agents and/or a bioactive polymer such as those described above can be placed in the fillable outer chamber 722 prior to use. The coagulated blood would then provide sufficient support for the tubular member or graft 712 in lieu of stents in the region surrounded by blood filled outer chamber 722. Leg 50 is shown attached to prosthesis 10. Any suitable method for attaching the leg to the bifurcated stent-graft can be used as would be apparent to one of ordinary skill in the art.

Referring to FIGS. 5E1-3, further variations are shown. Outer member 20 can extend to the suprarenal stent (FIG. 1) or spring stent (FIG. 2) adjacent to the suprarenal stent. This extension provides additional support for the graft all the way to those stents without including stents in the rejoin covered by outer member 20. Otherwise Prostheses 10′ and 100′ are the same as prosthesis 10 and 100. Accordingly, elements 10a′, 10b′, 12′, 18′, 20′, 20a′, 20b′, 22′, 24a′, 24b′, 26a′, 26b′, 51′, and S2a′-S2c′ as shown in FIG. 5E1 are the same as 10a, 10b, 12, 18, 20, 20a, 20b, 22, 24a, 24b, 26a, 26b, 51, and S2a-S2c as shown in FIG. 1._Similarly, elements 100a′, 100b′, 112′, 118′, 120′, 120a′, 120b′, 122′, 124a′,124b′, 126a′, 126b′, S5′, S6a′-S6e′, and S8′ as shown in FIG. 5E2 are the same as 100a, 100b, 112, 118, 120, 120a, 120b, 122, 124a, 124b, 126a, 126b, S5, S6a-S6e, and S8 as shown in FIG. 2. Elements 50′ (FIG. 5E1) and 150′ (FIG. 5E2) also are the same as elements 50 (FIGS. 1) and 150 (FIG. 2), respectfully. Therefore, S3a′-S3a′ are the same as S3a-S3c. FIG. 5E3 shows prosthesis 10′ deployed and in an inflated state.

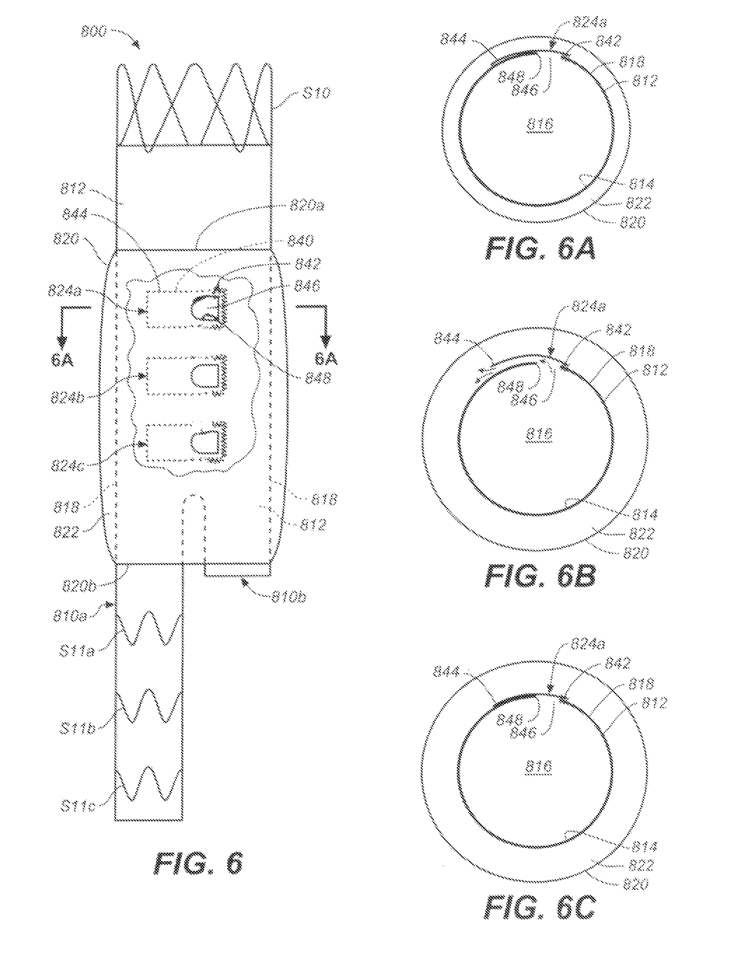

Referring to FIGS. 6, FIG. 6 illustrates another blood inflating prosthesis embodiment 800 which is the same as prosthesis 10 with the exception that outer member valves 24a-f and 26a-f are replaced with valves 824a,b,c. Additional valve sets similar to valves 824a,b,c can be circumferentially spaced there from in a manner similar to that described regarding blood inflating prosthesis 10. That is, prosthesis 800 can includes three six valve sets, each six valve set being circumferentially spaced as shown in FIGS. 1, 3A, and 3B in connection with prosthesis 10. Further, a different number of valves or other arrangements can be used as described in connection with prosthesis 10 as well. Otherwise, elements 810a, 810b, 812, 814, 816, 818, 820, 820a, 820b, and 822 are the same as elements 10a, 10b 12, 14, 16, 18, 20, 20a, 20b, and 22. Prosthesis 800 includes also undulating annular bare spring or crown stent S10, which can be a suprarenal stent, undulating annular stents S11a-c. Prosthesis 800 also can include optional undulating annular spring element or stent similar to S8 as shown in FIG. 2.

Each valve has the same construction. Therefore, description of the valves will be made regarding valve 824a only for purposes of simplification. Valve 824a is a one-way flap valve and comprises opening 846, which is formed through tubular member 812, which defines fluid/blood flow lumen 816, and is defined by opening perimeter 848, and valve member 840, which can be formed from the same material as the graft material of the prosthesis, which can be a stent-graft. Valve member 840 rests against tubular member 812 and covers opening 846 and opening perimeter 848 when in a relaxed state. Valve member 840 has a first portion 842 secured along its perimeter to the inner wall of tubular member 812 such as by stitching as shown with a zig-zag line. Alternatively, first portion 842 can be heat sealed to the inner wall of tubular member 812 along the illustrated stitched line. Valve member 840 further includes second portion 844, which makes up the remainder of the valve member and is not directly secured to tubular member 812 such that valve member 840 is free to pivot at one end of first portion 842 and move away from tubular member 812 and opening 846 when the relative pressure between inner lumen 816 and outer chamber 812 is as described above in connection with valves 24 and 26.

Referring to FIGS. 6A-C, sectional views taken along line 6A-6A in FIG. 6 are shown to illustrate valve operation during filling of chamber 822. FIG. 6A shows the valve closed and in sealing engagement with the outer surface 818 of tubular member 812. FIG. 6B illustrates the prosthesis being inflated with the depicted valve open with blood flowing from lumen 816 through opening 846 and into outer chamber 822 (see the arrow depiction). FIG. 6C illustrates prosthesis 800 inflated with the depicted valve closed as a result of the pressure in outer chamber 822.

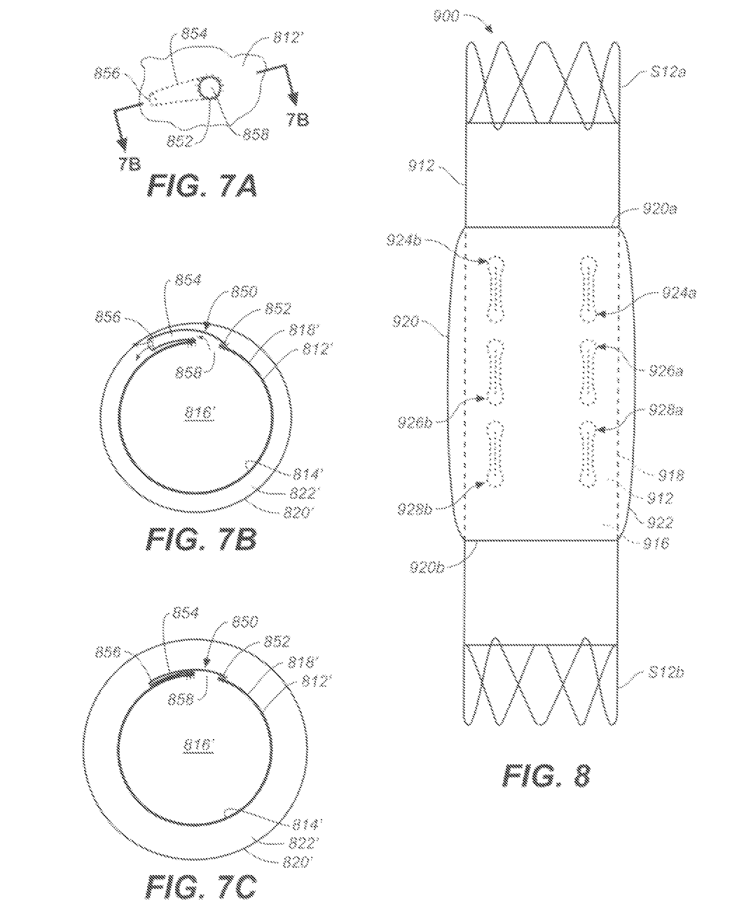

FIGS. 7A-C illustrate another one-way valve configuration that can be used in a prosthesis otherwise having the same construction as prosthesis 800 where tubular member 812′, inner surface 814′ of tubular member 812′, inner lumen 816′, outer member 820′, and outer chamber 822′ are the same as tubular member 812, inner surface 814, inner lumen 816, outer member 820, and outer chamber 822. Valve 850 can correspond to a duckbill valve or flutter valve comprising a tubular member or valve member 854, which can be made from the same graft material as prosthesis 10 or 800, and opening 858 formed through tubular member 812′ in a similar manner that opening 846 is formed in tubular member 812. Tubular member 854 has one end 852 that forms an opening and is secured to the outer surface of tubular member 812′ around tubular member opening 858 to form a fluid seal therebetween. This can be done by stitching as shown with the zig-zag line. Alternatively, it can be heat sealed to tubular member 812′ to form a fluid tight seal or a combination of stitching and heat sealing, which can be used on any of the seals described herein. Tubular member 854 also has a free end 856 from which fluid can flow into outer chamber 822′. Free end 856 is not directly secured to tubular member 812′. In the case where valve 850 is a one-way duckbill valve, tubular member 854 is flat and closed when in a relaxed state and opens into a tubular shape when fluid under sufficient pressure passes into the opening of end 852, through tubular member 854 and out the opening of free end 856. However, when valve 856 is a one-way flutter valve, which operates like a duckbill valve, except that one side of the bill is formed by the substrate to which the bill shaped member of the is attached, thus the one side (membrane) flutters when passing fluid and seals tight against the substrate, when a reverse flow or pressure is attempted. In use, blood flows (as depicted with the arrows in FIG. 7B) from lumen 816, through opening 858, through tubular member 854, which can be cylindrical or tapered when in an open configuration, and out from the open free end 856 of tubular member 854 when the relative pressures in lumen 816′ and outer chamber 822′ are as described above in connection with valves 24 and 26. After outer chamber 822′ has been filled, tubular member 854 flattens in the case where the valve is a duckbill valve due to an insufficient pressure differential between inner lumen 816′ and outer chamber 822′ (FIG. 7C). However, in the case where the valve is a flutter valve the pressure in chamber 822′ flattens tubular member 854 to close the valve (FIG. 7C).

Referring to FIG. 8, another blood inflating prosthesis embodiment is shown and designated with reference numeral 900. Blood inflating prosthesis 900 is which is the same as prosthesis 10 with the exception tubular member 912 is a single lumen member having an optional second bare wire stent S12b at the opposite end of tubular member 912 where suprarenal stent S12a is positioned, three rows of valves are shown as compared to two rows, and outer member 920, which can be made of any of the materials described above in connection with outer member 20, is a tubular member having two open ends prior to attachment to tubular member 912. Accordingly, valves 924a,b, 926a,b, and 928a,b have the same construction as valves 24 and 26 of prosthesis 10 and are formed in tubular member 912 in the same manner as valves 24 and 26 are formed in tubular member 12. Further, additional valves can be circumferentially spaced around tubular member 912 in the same manner as described above in connection with prosthesis 10. It also should be understood that other valve constructions can be used such as those shown in FIGS. 6, 6a-c, and 7a-c. Further, any of the valves described above can be used in any of the embodiments described herein. The arrangement and number of valves also can vary as would be apparent to one of ordinary skill in the art.

Regarding attachment of outer member of 920 to inner tubular member 12, which forms inner lumen 916 through which blood flows, tubular outer member 920 is attached to outer surface 918 of tubular member 912 to form a fluid tight seal between annular ends 920a and 920b of outer member 912 and tubular member 912 and provide a fillable chamber 922 formed by a portion of tubular member 912 and outer member 922. The fluid tight seal can be made using any suitable sealing means such as thermal/heat sealing, adhesive bonding or gluing, or any combination thereof. Outer member 920 can be pre-molded as a tubular member or it can be formed from a sheet where side portions are sealed together to form a longitudinal seam using any of the sealing means described above.

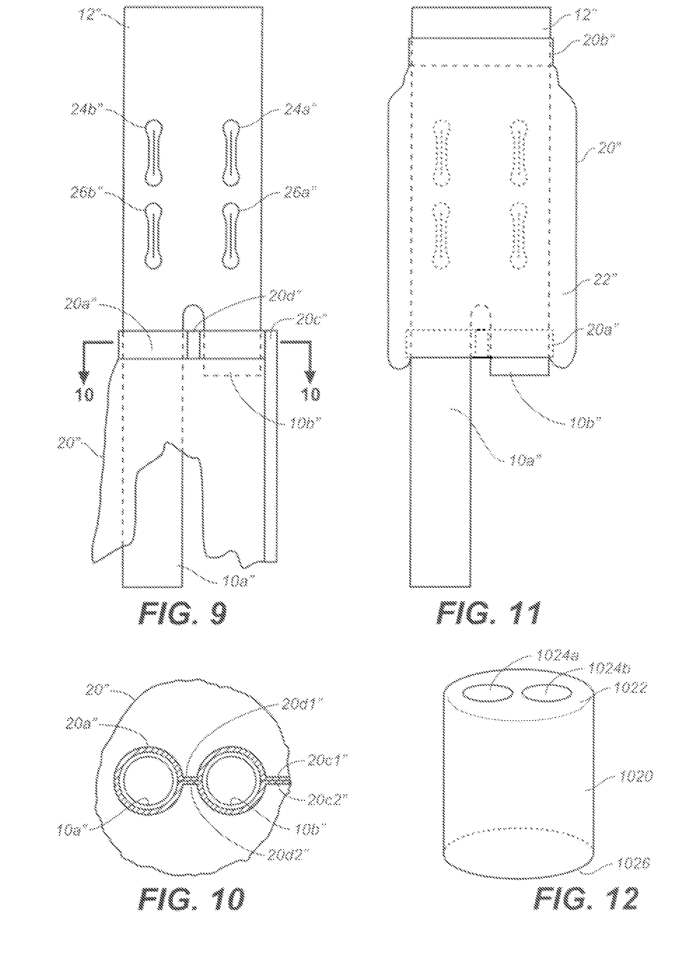

Referring to FIGS. 9-11, another method of assembling an outer member with a bifurcated graft is shown. In this example, tubular member 12″ is the same as tubular member 12 and includes the same valves, which in this view are indicated with reference numerals 24a″, 24b″, 26a″, and 26b″. In this method of assembly, a flat sheet of material (e.g., compliant material) forms outer member 20″ and can comprise the same compliant material(s) as outer member 20 described above. Flat sheet 20″ is wrapped around legs 10a″ and 10b″ and end portion 20a″ of sheet 20″ secured thereto to form a fluid tight seal therebetween by any suitable sealing means such thermal/heat sealing, adhesive bonding or gluing, or any combination thereof (FIGS. 9-10). As shown in FIG. 10, overlapping mid-sections 20d1″ and 20d2″ of end portion 20a″ between legs 10a″ and 10b″ are sealed together to form a fluid tight seal therebetween using any suitable sealing means such as those described above. Further, the longitudinal side margins 20c1″ and 20c2″of the sheet are then secured together to form a fluid tight seal 20c″ (FIG. 9) therebetween using any suitable sealing means such as those described above. In this manner a generally cylindrical member with an open end and a closed end sealingly secured to tubular member 12″ is formed.

Referring to FIG. 11, the generally cylindrical member, which forms outer member 20″, is everted and pulled back over the body of tubular member 12″ and its other end portion 20b″ is secured to tubular member 12″ to form a fluid tight seal therebetween using any suitable sealing means such as those described above (e.g., with a heat seal or an adhesive or glue). In this manner, fillable outer chamber 22″ is formed by and between tubular member 12″ and outer member 22″.

Referring to FIG. 12, a further alternative outer member 1020 is shown and which corresponds to outer member 20 except that the cylinder diameter of member 1020 is shown with a constant diameter. Outer member 1020 can comprise the same material as outer member 20. Outer member 1020 has a cylindrical configuration and has a first end formed by disk portion 1022 and a second end 1026 forming an opening for receiving the single lumen portion of tubular member 12 or any corresponding tubular member as described above. Disk portion 1022 has two holes 1024a,b formed therethrough for receiving legs 10a and 10b or any corresponding legs as described above.

Any feature described in any one embodiment described herein can be combined with any other feature or features of any of the other embodiments or features described herein. Furthermore, variations and modifications of the devices and methods disclosed herein will be readily apparent to persons skilled in the art.

What is claimed is:

1. A tubular prosthesis comprising:
a tubular member that defines an inner lumen, said tubular member having an inner surface and an outer surface;
an outer member secured to said tubular member, said outer member covering at least a portion of said tubular member outer surface and forming an outer chamber therewith; and
at least one valve in said tubular member to control fluid flow between said tubular member inner lumen and said outer chamber.

2. The tubular prosthesis of claim 1, wherein said at least one valve is a one-way valve oriented to allow fluid flow from said inner lumen to said outer chamber.

3. The tubular prosthesis of claim 2, including a plurality of valves in said tubular member, each oriented to provide controlled fluid flow between said inner lumen and said outer chamber.

4. The tubular prosthesis of claim 3, wherein said plurality of valves are circumferentially spaced around said tubular member.

5. The tubular prosthesis of claim 3, where said plurality of valves are longitudinally spaced along said tubular member.

6. The tubular prosthesis of claim 5, wherein said plurality of valves are circumferentially spaced around said tubular member and a plurality of said one-way valve are longitudinally spaced along said tubular member.

7. The tubular prosthesis of claim 1, wherein said outer member surrounds said tubular member and forms a 360 degree chamber therewith.

8. The tubular prosthesis of claim 1, including a plurality of valves in said tubular member and a plurality of outer members, each outer member covering a portion of said tubular member and at least one valve to form a chamber with said tubular member.

9. The tubular prosthesis of claim 8, wherein said plurality of outer members are arranged to form a plurality of ribs when filled with fluid.

10. The tubular prosthesis of claim 9, wherein said plurality of outer members are arranged to form a plurality of longitudinally extending and circumferentially spaced ribs.

11. The tubular prosthesis of claim 1, wherein said valve comprises an openable portion and a spring element surrounding said openable portion.

12. The tubular prosthesis of claim 11 wherein said openable portion comprises a slit formed in said tubular member.

13. The tubular prosthesis of claim 12 wherein said spring element expands when the pressure differential in said inner lumen and outer chamber exceeds a certain value.

14. The tubular prosthesis of claim 1, further in including a crown stent secured to one end of the tubular prosthesis.

15. The tubular prosthesis of claim 14, further comprising a main body portion and a leg extending therefrom, said main body portion having a diameter greater than the diameter of said leg and being unsupported by stents except for said crown stent.

16. The tubular prosthesis of claim 15, further including at least one stent supporting said leg.

17. The tubular prosthesis of claim 1, wherein said tubular member is unsupported by stents under said outer member.

18. The tubular prosthesis of claim 1, wherein a blood coagulation agent is disposed in said outer chamber.

19. The tubular prosthesis of claim 2, wherein said at least one valve is a flap valve.

20. The tubular prosthesis of claim 2, wherein said at least one valve comprises a tubular member having an open tubular configuration and a closed configuration.

21. The tubular prosthesis of claim 14, wherein said outer member extends to said crown stent and said tubular member is unsupported by stents under said outer member.

22. The tubular prosthesis of claim 14, further including a second stent adjacent to said crown stent and wherein said outer member extends to said second stent and said tubular member is unsupported by stents under said outer member.

23. The tubular prosthesis of claim 1, further including a blood coagulating agent or a bioactive biopolymer disposed in said outer chamber.
